(19) United States
(12) Patent Application Publication
(10) Pub. No.: US 2008/0188688 A1
(43) Pub. Date: Aug. 7, 2008

(54) SOLVENTS FOR PHASE-TRANSFER-CATALYZED REACTIONS

(76) Inventors: Hendrik Huesken, Monheim am Rhein (DE); Paul Birnbrich, Sollngen (DE); Rainer Hoefer, Duesseldorf (DE)

Correspondence Address: COGNIS CORPORATION;PATENT DEPARTMENT, 300 BROOKSIDE AVENUE, AMBLER, PA, 19002, US

(21) Appl. No.: 12/024,363

(22) Filed: Feb. 1, 2008

(30) Foreign Application Priority Data
Feb. 2, 2007 (DE) 10 2007 005 283.0

(51) Int. Cl. C07C 45/63

(52) U.S. Cl. 568/316

(57) ABSTRACT

A process for the production of organic substances, including the steps of providing at least a two-phase system, in which selected reactants are reacted in the presence of a phase transfer catalyst, where one of the phases is an organic solvent with a log P value, at 20° C., of more than 2.6, is provided.

### CROSS-REFERENCE TO RELATED APPLICATIONS

This application claims priority under 35 U.S.C. § 119 from German Patent Application No. 10 2007 005 283.0, filed Feb. 2, 2007, the entire disclosure of which is hereby incorporated by reference herein.

### BACKGROUND OF THE INVENTION

1. Field of the Invention

The invention relates generally to the use of solvents for phase-transfer-catalyzed reactions, and more particularly, to organic substances with a log P value of more than 2.6 as solvents for reactions accelerated by phase transfer catalysts. The invention also relates to a process for the production of organic substances in which the reactants are reacted with one another in the presence of a phase transfer catalyst in a system containing at least two phases, one of the phases being an organic solvent which has a log P value of more than 2.6.

2. Background Information

“Phase transfer catalysis” (referred to as PTC) is an important method in organic synthesis in which an anionic reagent is generally transferred from the aqueous or solid phase into an organic phase. The transferred reagent then has a distinctly increased reactivity level for the intended reaction by virtue of the lower hydration, the increased concentration and the greater proximity to the reactant. Common reagents which can be transferred include:
a) bases, such as OH⁻, HCO₃⁻,
b) nucleophiles, such as fluoride, chloride, bromide, iodine, CN⁻, R⁻, RCO₂⁻,
c) oxidizing agents, such as permanganate and perchromate, and
d) reducing agents, such as BH₄⁻.

The advantages of PTC lie above all in higher yields, milder reaction conditions, fewer impurities and easier working up of the reaction mixture.

Aqueous sodium hydroxide is widely used. It can replace alcoholates, amides and hydrides under phase transfer conditions. Nucleophilic substitutions, dehydrohalogenations, oxidations and reductions and many other reactions, including, for example, reactions important for the production of polymers, are accessible to PTC. The mechanism of nucleophilic substitution by PTC can be explained by the following example of the commonly used quaternary ammonium cations:

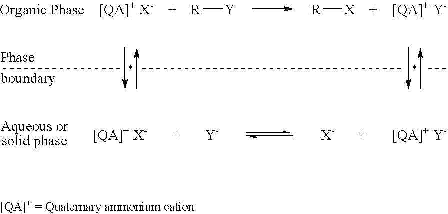

The ammonium cation forms an ion pair with the reagent X⁻ for which a concentration equilibrium is established between the two phases. X⁻ then reacts rapidly in the organic phase with the reactant R—Y to form the product R—X and a new ion pair of Y⁻ with the ammonium cation. A concentration equilibrium between the two phases is also established for this new ion pair, the catalyst in the aqueous or solid phase exchanging the Y⁻ split off for a new reagent X⁻ so that a new catalysis cycle can begin.

PTC-catalyzed reactions typically take place in a two-phase system, of which one phase is water or a solid, and the other phase is a water-insoluble reactant. Since the reaction temperature selected for reactions carried out under phase transfer catalysis conditions can often be extremely low (typically <80° C.), a solvent is often required to dissolve a water-insoluble educt, if, for example, it has a melting point above the reaction temperature. The use of a solvent generally affords the following advantages:
it enables reactants with a high melting point to be used
it enables the reaction to be carried out at a temperature below the melting point
the viscosity of the educt and/or product and hence of the system as a whole can be reduced where necessary
it enables hydrolysis-sensitive substances to be reacted in the presence of water.

Numerous examples where a solvent is required in phase transfer catalysis (PTC) can be found in the literature for all types of reactions. Typical solvents include toluene, benzene, MIBK, MTBE, xylene and dichloromethane. However, in view of the potential danger of these solvents, they are unsuitable for use in industrial processes and in laboratory experiments. Although examples in the PTC field where no solvents are used can also be found in the literature, the use of a solvent is absolutely essential in many cases.

There remains a need for suitable solvents for use in phase-transfer catalzyed reactions.

### SUMMARY OF THE INVENTION

Briefly described, according to an aspect of the invention, a process for the production of organic substances, includes the steps of providing at least a two-phase system, in which selected reactants are reacted in the presence of a phase transfer catalyst, where one of the phases is an organic solvent with a log P value, at 20° C., of more than 2.6.

### DETAILED DESCRIPTION OF THE INVENTION

The problem addressed by the present invention was to provide solvents for phase-transfer-catalyzed reactions (PTC reactions). These solvents would provide for the optimal management of PTC reactions (high reaction rates and good yields). In addition, they would pose a lower risk potential to humans and the environment by comparison with conventional solvents, such as toluene, benzene, MIBK, MTBE, xylene and dichloromethane.

The present invention relates to the use of organic substances X with a log P value of more than 2.6 as solvents for reactions accelerated by phase transfer catalysts, the log P value being experimentally determined in accordance with OECD Guideline No. 107 for the Testing of Chemicals.

The log P value of an organic substance X is defined as follows:

log Pₓ=log [(c_{X(water)})/(c_{X(n-octanol)})]

where c_{water} and c_{n-octanol} respectively represent the equilibrium concentration which is established when the organic substance X is introduced into the binary system of water and n-octanol (present in a ratio by weight of 1:1) at 20° C. and “log” is the decadic logarithm.

In principle, the log P value of a substance X is determined by introducing the organic substance X into a 1:1 mixture (by weight) of water and n-octanol at 20° C., waiting until the equilibrium is established and then determining the equilibrium concentration of X in water and n-octanol. The quotient of the equilibrium concentrations is then formed and the decadic logarithm is taken from that quotient.

It is expressly pointed out that, for the purposes of the present invention, the log P value is experimentally determined in accordance with OECD Guideline No. 107 for the Testing of Chemicals (dated 27 Jul. 1995).

The log P value of the solvents to be used in accordance with the invention is preferably above 2.6 and more particularly above 2.8.

In a preferred embodiment, substances with a log P value above 2.8 selected from the group of fatty acid dialkyl amides, more particularly fatty acid dimethyl amides, are used in accordance with the invention as solvents for PTC reactions. Fatty acids in the present context are understood to be carboxylic acids containing 8 to 24 carbon atoms which are preferably saturated. As can be seen from the Examples of the present specification, fatty acid dimethyl amides are not only equivalent, but superior to conventional solvents in regard to the reaction rates in PTC reactions. Examples of suitable fatty acid dimethyl amides are octanoic acid dimethyl amide, decanoic acid dimethyl amide, dodecanoic acid dimethyl amide and tetradecanoic acid dimethyl amide. In another embodiment, fatty acid dimethyl amides derived from a branched, saturated fatty acid are used as solvents. Mixtures of various fatty acid dimethyl amides may also be used.

In another embodiment, substances with a log P value above 2.6 (and preferably above 2.8) selected from the group of carboxylic acid alkyl esters (carboxylic acid methyl, ethyl or isopropyl ester being particularly preferred), glycerol, dicarboxylic acid esters and ethyl lactate are used as solvents for PTC reactions.

The solvent to be used in accordance with the invention should be inert to the reaction conditions of the desired PTC reaction.

In a preferred embodiment, the solvent to be used in accordance with the invention has a melting point below 20° C.

### Phase Transfer Catalysts

Phase transfer catalysts in the context of the present invention are organic substances which accelerate the reaction of the reactants to form the compounds to be produced. The reaction system has at least two phases.

The phase transfer catalysts used may be virtually any of the phase transfer catalysts known to the expert for this purpose. Suitable examples of suitable phase transfer catalysts are the groups of tetraalkyl ammonium salts, benzyl trialkyl ammonium salts, tetraalkyl phosphonium salts, benzyltrialkyl phosphonium salts and mixtures thereof, polyethylene glycols and end-capped polyethylene glycols.

In addition, substances known to the expert by the name of ionic liquids are also suitable for use as phase transfer catalysts. Ionic liquids are understood by convention to be organic salts which have a melting point below 100° C. Ionic liquids which have a melting point below 20° C. are preferably used as phase transfer catalysts.

The ammonium salts are preferred to the phosphonium salts for the purposes of the present invention, the tetra-n-butyl ammonium, tri-n-butyl methyl ammonium and benzyl triethyl ammonium salts with the anions chloride, bromide and hydrogen sulfate being particularly suitable. A most particularly preferred phase transfer catalyst is the trioctyl methyl ammonium chloride commercially obtainable under the name of ALIQUAT® 336 (manufacturer: Cognis). Another phase transfer catalyst preferred for the purposes of the present invention is ALIQUAT® HTA-1 (manufacturer: Cognis).

The phase transfer catalysts are used in catalytic quantities. The quantity depends on the activity and stability of the catalyst under the selected reaction conditions and has to be adapted to the particular reaction. The quantity in which the phase transfer catalysts are used can vary between 0.1 and 25 mol-%, based on the sum of the reactants, and is preferably between 0.5 and 5 mol-% and more particularly between 1 and 3 mol-%.

### Carrying Out the Phase Transfer Catalysis

Basically, the conduct of PTC reactions is not subject to any particular limitations. The reaction system has at least two phases.

In one embodiment, the reactions are carried out continuously.

In another reaction, these reactions are carried out discontinuously (in batches).

More than one liquid phase is present in the PTC reactions. In a variant, three phases are present. One liquid phase is often an aqueous phase. This aqueous phase may also be present on a solid as a so-called omega phase.

In a particularly preferred embodiment, two phases are present, preferably an aqueous phase and an organic phase. The two phases may be used in quantity ratios of 90:10 to 10:90 (by weight).

On completion of the PTC reaction, working up may be carried out by any of the separation and purification techniques known to the expert for this purpose. If the organic substance produced is insoluble in water and is dissolved in the ionic liquid, it can be recovered therefrom by distillation-based methods or by extraction with supercritical carbon dioxide.

The present invention also relates to a process for the production of organic substances in which the reactants are reacted with one another in a system containing at least two phases in the presence of a phase transfer catalyst, characterized in that one of the phases is an organic solvent which has a log P value of more than 2.6. All the foregoing observations regarding the solvent otherwise apply to the process according to the invention.

The process according to the invention is not subject to any limitations in regard to the nature of the PTC-catalyzed reaction. The process may be applied to all conventional PTC reactions, including for example O alkylation, C alkylation, N alkylation, S alkylation, esterification, transesterification, oxidation, reduction, oxidation, Michael addition, aldol condensation, condensation reactions, dehydrohalogenations, hydrolyses, epoxidations, carbonylation reactions, chiral reactions. Reactions involving hydrolysis-sensitive substances are also possible.

As already mentioned, the solvent to be used in accordance with the invention should be inert to the reaction conditions of the desired PTC reaction.

The solvent to be used in accordance with the invention preferably has a melting point below 20° C.

### EXAMPLES

Log P values were experimentally determined in accordance with OECD Guideline 107 for the Testing of Chemicals (dated 27 Jul. 1995).

The measured data are set out in the following Table:

| | Log P |
|---|---|
| | determined in accordance with OECD |
| Substance/solvent | Guideline 107 for the Testing of Chemicals |
| MTBE | 1.12 |
| MIBK | 1.40 |
| Toluene | 2.49 |
| Benzene | 2.09 |
| Decanoic acid dimethyl | 2.94 |
| amide | |
| Decanoic/octanoic acid | 2.83 |
| dimethyl amide | |

### ABBREVIATIONS

MTBE=methyl tert.butyl ether; log P value 106
MIBK=methyl isobutyl ketone; log P value 1.31
Decanoic acid=AGNIQUE® KE 3308 (Cognis); log P value 2.94 dimethyl amide
Decanoic/octanoic=AGNIQUE® KE 3658 (Cognis); log P value 2.83 acid dimethyl amide
ALIQUAT® 336=phase transfer catalyst (Cognis)

### Example 1

### Comparison

### (C Alkylation of Deoxybenzoin in the Presence of MTBE as Solvent)

Deoxybenzoin (18.83 g, 96 mmol) was introduced into a nitrogen-purged reactor (reactor volume about 300 ml) together with a five-fold molar excess of 50% sodium hydroxide (53.82 g, 480 mmol) and MTBE (53.82 g). n-Decane (3.30 g) was then added (as internal standard for GC evaluation). The mixture was heated to a reaction temperature of 45° C. with continuous stirring at a stirrer speed of 500 r.p.m. 5.5 g ALIQUAT® 336 (6 mol-%, based on deoxybenzoin) and 14.16 g (=120 mmol-%, based on deoxybenzoin) isopropyl bromide were added at 45° C. to start the reaction. The total quantity of reaction mixture was 150 g which filled about half the reactor. The conversion was determined by gas chromatography (GC). Result: conversion after 90 mins.: 28%.

### Example 2

### Comparison

### (C Alkylation of Deoxybenzoin in the Presence of MIBK as Solvent)

Deoxybenzoin (18.83 g, 96 mmol) was introduced into a nitrogen-purged reactor (reactor volume about 300 ml) together with a five-fold molar excess of 50% sodium hydroxide (53.82 g, 480 mmol) and MIBK (53.82 g). n-Decane (3.30 g) was then added (as internal standard for GC evaluation). The mixture was heated to a reaction temperature of 45° C. with continuous stirring at a stirrer speed of 500 r.p.m. 5.5 g ALIQUAT® 336 (6 mol-%, based on deoxybenzoin) and 14.16 g (=120 mmol-%, based on deoxybenzoin) isopropyl bromide were added at 45° C. to start the reaction. The total quantity of reaction mixture was 150 g which filled about half the reactor. The conversion was determined by gas chromatography (GC). Result: conversion after 90 mins.: 75%.

### Example 3

### Invention

### (C Alkylation of Deoxybenzoin in the Presence of Decanoic Acid Dimethyl Amide as Solvent)

Deoxybenzoin (18.83 g, 96 mmol) was introduced into a nitrogen-purged reactor (reactor volume about 300 ml) together with a five-fold molar excess of 50% sodium hydroxide (53.82 g, 480 mmol) and decanoic acid dimethyl amide (53.82 g). n-Decane (3.30 g) was then added (as internal standard for GC evaluation). The mixture was heated to a reaction temperature of 45° C. with continuous stirring at a stirrer speed of 500 r.p.m. 5.5 g ALIQUAT® 336 (6 mol-%, based on deoxybenzoin) and 14.16 g (=120 mmol-%, based on deoxybenzoin) isopropyl bromide were added at 45° C. to start the reaction. The total quantity of reaction mixture was 150 g which filled about half the reactor. The conversion was determined by gas chromatography (GC). Result: conversion after 90 mins.: 98%.

### Example 4

### Invention

### C Alkylation of Deoxybenzoin in the Presence of Decanoic/Octanoic Acid Dimethyl Amide as Solvent

Deoxybenzoin (18.83 g, 96 mmol) was introduced into a nitrogen-purged reactor (reactor volume about 300 ml) together with a five-fold molar excess of 50% sodium hydroxide (53.82 g, 480 mmol) and decanoic/octanoic acid dimethyl amide (53.82 g). n-Decane (3.30 g) was then added (as internal standard for GC evaluation). The mixture was heated to a reaction temperature of 45° C. with continuous stirring at a stirrer speed of 500 r.p.m. 5.5 g ALIQUAT® 336 (6 mol-%, based on deoxybenzoin) and 14.16 g (=120 mmol-%, based on deoxybenzoin) isopropyl bromide were added at 45° C., to start the reaction. The total quantity of reaction mixture was 150 g which filled about half the reactor. The conversion was determined by gas chromatography (GC).

Result: conversion after 90 mins.: 98%.

### Example 5

### Comparison

### (C Alkylation of Deoxybenzoin in the Presence of MTBE as Solvent Without a Phase Transfer Catalyst)

Deoxybenzoin (18.83 g, 96 mmol) was introduced into a nitrogen-purged reactor (reactor volume ca. 300 ml) together with a five-fold molar excess of 50% sodium hydroxide (53.82 g, 480 mmol) and MTBE (53.82 g). n-Decane (3.30 g) was then added (as internal standard for GC evaluation). The mixture was heated to a reaction temperature of 45° C. with continuous stirring at a stirrer speed of 500 r.p.m. 14.16 g (=120 mmol-%, based on deoxybenzoin) isopropyl bromide were added at 45° C. to start the reaction. The total quantity of reaction mixture was 150 g which filled about half the reactor. The conversion was determined by gas chromatography (GC). Result: conversion after 90 mins.: 0%.

### Summary of the Results of the Examples:

| Example | | | Log | Conversion²⁾ |
|---|---|---|---|---|
| No. | Solvent | Catalyst | P¹⁾ | [%] |
| 1 | MTBE | ALIQUAT ® 336 | 1.12 | 28 |
| 2 | MIBK | ALIQUAT ® 336 | 1.40 | 75 |
| 3 | Decanoic acid | ALIQUAT ® 336 | 2.94 | 98 |
| | dimethyl amide | | | |
| 4 | Decanoic/ | ALIQUAT ® 336 | 2.83 | 98 |
| | octanoic acid | | | |
| | dimethyl amide | | | |
| 5 | MTBE | None | 1.12 | 0 |

¹⁾Log P value of the solvent (as measured in accordance with Guideline No. 107 for the Testing of Chemicals dated 27.07.95)
²⁾Conversion after 90 minutes

What is claimed is:

1. A process for the production of organic substances, comprising the steps of:
providing at least a two-phase system, in which selected reactants are reacted in the presence of a phase transfer catalyst, wherein one of the phases is an organic solvent with a log P value, at 20° C., of more than 2.6.

2. The process according to claim 1, wherein the two-phase system comprises a water phase, and an organic solvent with a log P value of more than 2.62.

3. The process according to claim 2, wherein the organic solvent comprises a saturated fatty acid dimethyl amide.

4. The process according to claim 1, wherein the organic solvent has a melting point below 20° C.

5. The process according to claim 1, wherein the organic solvent has a log P value of more than 2.8.

6. The process according to claim 1, wherein the phase transfer catalyst is selected from the group consisting of: tetraalkyl ammonium salts, benzyl trialkyl ammonium salts, tetraalkyl phosphonium salts, benzyltrialkyl phosphonium salts, polyethylene glycols and end-capped polyethylene glycols.

7. The process according to claim 2, wherein the phase transfer catalyst is selected from the group consisting of: tetraalkyl ammonium salts, benzyl trialkyl ammonium salts, tetraalkyl phosphonium salts, benzyltrialkyl phosphonium salts, polyethylene glycols and end-capped polyethylene glycols.

8. The process according to claim 3, wherein the phase transfer catalyst is selected from the group consisting of: tetraalkyl ammonium salts, benzyl trialkyl ammonium salts, tetraalkyl phosphonium salts, benzyltrialkyl phosphonium salts, polyethylene glycols and end-capped polyethylene glycols.

9. The process according to claim 1, wherein the phase transfer catalyst is an ionic liquid.
